(19) United States
(12) Patent Application Publication
(10) Pub. No.: US 2005/0009117 A1
(43) Pub. Date: Jan. 13, 2005

(54) Diagnostic kit and method using phycoerythrin to label tumor tissues and cells

(75) Inventors: Chuang-Chun Chiueh, Taipei (TW); Err-Cheng Chan, Taoyuan County (TW); Chia-Rui Shen, Taoyuan County (TW)

Correspondence Address: BACON & THOMAS, PLLC, 625 SLATERS LANE, FOURTH FLOOR, ALEXANDRIA, VA, 22314, US

(73) Assignee: Far East Biotec Co., Ltd., Taipei (TW)

(21) Appl. No.: 10/682,192

(22) Filed: Oct. 10, 2003

(30) Foreign Application Priority Data
Jul. 8, 2003 (TW) 92118592

(51) Int. Cl. C12Q 1/68; G01N 33/574; G01N 1/30; G01N 33/48

(52) U.S. Cl. 435/7.23; 435/40.5

(57) ABSTRACT

This invention provides a method and a diagnostic kit that utilizes phycoerythrin to label tumor cells, wherein the high specificity and high affinity of phycoerythrin is to be used to label and enter tumor cells. With effective amount of phycoerythrin and suitable wavelength of light, the stimulated fluorescence from phycoerythrin can be used for tumor diagnosis.

### BACKGROUND OF THE INVENTION

(A) Field of Invention

This invention provides a method and diagnostic kits using Phycoerythrin to label tumor cells. Specifically, phycoerythrin is introduced to tumor cells first and its high specificity and affinity to tumor cells were utilized together with various selected wavelength of lights to stimulate phycoerythrin so as to generate fluorescence for tumor diagnosis.

(B) Description of Prior Arts

Each year there are millions of people died of cancer all over the world. In Taiwan cancer is ranked the number one on the list of top ten causes of death. Since most cancer patients seek medical advises only after their symptoms turned significant which usually means terminal stage already. If cancer can be diagnosed on its early stage, then it would be helpful to patients in their treatment and prognosis. Recently quite a few tumor screening kits have been developed, most of them use specific antibody to identify relevant tumor markers (table 1), therefore, their effectiveness is heavily rely on the characteristics of tumor markers.

TABLE 1
Common tumor markers for cancer screening and their clinical
applications

| Screening markers | Clinical applications |
|---|---|
| Carcino-embryonic antigen | Screening of early stage cancer cells |
| | Colon cancer, rectal cancer; digestive |
| | cancer |
| Alpha-fetoprotein | Screening of early stage cancer. |
| | Hepatoma |
| CA199 | Pancreatic cancer, colon cancer; |
| | gastric cancer; Cholangiocarcinoma |
| Ferritin | Lung cancer; leukemia |
| PSA | Prostate and Genitourinary Cancer |
| CA125 | Ovarian cancer or Hysteromyoma |
| CA153 | Breast cancer |
| Anti-Ebvirus in blood | Nasopharyngeal carcinoma |

In theory, an ideal tumor marker should carry following characteristics: 1) specificity, in other words, negative result should be obtained from healthy patient; 2) high sensitivity, and it is preferable that tumor be detected at the early stage; 3) tumor marker should be proportional to the size of tumor, which can be used to distinguish respective tumor stage, and also served as prognosis index or as a tracer to monitor effectiveness of treatment and recurrence; 4) the assay procedure must be definite, simple, convenient and reproducible .

However, practically most of tumor markers can barely reach such level. A substantial number of tumor markers increase even on benign tumors or other benign lesions. Besides, some tumor markers do not necessarily carry organ specificity. There are still some relevant tumor markers of certain tumors left undiscovered, thus causing diagnostic blindspot regarding tumor diagnosis.

In view of the above-mentioned drawbacks of previous tumor diagnosis technology, if one can develop a substance with high specificity and affinity to tumor cells for tumor identification, then such said substance will contribute significantly on tumor diagnosis and treatment.

### SUMMARY OF THE INVENTION

Due to the fact of technological shortcomings on tumor diagnosis, the present invention provides a method for labeling tumor tissues or cells by utilizing phycoerythrin. The method comprises the steps of: adding effective amount of phycoerythrin to tissues or cells and incubating for a certain period; exposing above said tissues or cells containing effective amount of phycoerythrin with appropriate wavelength of light to stimulate phycoerythrin to emit fluorescence; and monitoring above said fluorescence to identify the location and range of tumor tissues or cells.

The above said phycoerythrin comprises at least R-phycoerythrin or B-phycoerythrin, wherein R-phycoerythrin is more preferable.

The length of above said certain period of incubation varies according to cell types, generally from 0.5 hour to 24 hours.

The above said appropriate wavelength of light is between 488 to 633 nm, wherein the ideal wavelength is at 488 nm for R-phycoerythrin and 543 nm for B-phycoerythrin.

The above said effective amount of phycoerythrin is between 0.5 to 40 ug/mI, wherein the preferable effective amount is between 0.5 to 10 ug/mI.

The above said fluorescence is monitored at wavelength above 565 nm.

The above said tumor can be solid or liquid tumor, which comprises lymphoma, gastric cancer, hepatoma, colon cancer, lung fibroma, thyroid cancer, cervical carcinoma and brain tumor.

The above said method for labeling tumor tissues or cells by utilizing phycoerythrin wherein labeling tumor tissues or cells means that the tumor-recognizing and tumor-entering ability of phycoerythrin is utilized in order to achieve the performance of labeling tumor tissues or cells.

The above said method for labeling tumor tissues or cells by utilizing phycoerythrin can further comprise a step of analyzing the level of fluorescent staining of tumor so as to judge the stage of tumor. The above said analyzing the level of fluorescent staining of tumor can be achieved through immunofluorescence staining or flow cytometry.

The present invention also provides a diagnostic kit for labeling and locating tumor tissues or cells that comprises effective amount of phycoerythrin.

The above said phycoerythrin comprises R-phycoerythrin or B-phycoerythrin, wherein R-phycoerythrin is more preferable.

The present invention further provides a method for labeling tumor tissues or cells by utilizing phycoerythrin that can be achieved through above said diagnostic kits.

### BRIEF DESCRIPTION OF DRAWINGS

FIG. 1 shows that both R-phycoerythrin and B-phycoerythrin can enter cervical cancer cells.

FIG. 2 shows that phycoerythrin can selectively enter cancer cells rather than normal cells.

FIG. 3 shows phycoerythrin can enter leukemic cells from blood sample of leukemia patient.

FIG. 4 shows the immunofluorescence staining result that demonstrates B-phycoerythrin selectively enters cancer cells.

### DETAILED DESCRIPTION OF PREFERRED EMBODIMENT

The phycoerythrin of present invention, which comprises R-phycoerythrin and B-phycoerythrin, is a water-soluble, fluorescent pigment protein existed exclusively in algae. The R-phycoerythrin is a more preferable example of the present invention, and its major physical and chemical properties are as follows: 1) Its structure consists of three types of subunits, α`β and γ, which usually appear in hexamer form of (αβ)₆γ. 2) Its molecular weight is 260,000 dalton. 3) Its absorption spectrum falls within visible light range, wherein the primary absorbance peak is at 566 nm with a secondary peak at 498 nm. 4) The fluorescence emission spectrum falls within visible light range, wherein the peak of the fluorescence wavelength is at 575 nm. 5) The intensity of fluorescence is strong and stable with rarely affected by pH, temperature and other biomolecules. 6) Its maximum excitation wavelength is at 488 nm, with fluorescence quantum yield of 0.84. 7) It contains specific phycobilin that is bound covalently to a homocysteine resdue of the pigment protein. 8) It has good water solubility and long, stable shelf life when exists in the states of solution or ammonium sulfate precipitate.

The present invention utilizes the tumor-recognizing and tumor-entering ability of phycoerythrin so as to achieve the performance of labeling tumor tissues or cells. There is no limitation in terms of types of tumor to be labeled, however, the amount of phycoerythrin, as well as the intensity and duration of illumination would be variable respective to different types of tumor.

The following is a detailed description of the best presently known modes of carrying out the inventions. This description is not to be taken in a limiting sense, but is made merely for the purpose of further illustrating the present inventions.

### EXAMPLE 1

### Phycoerythrin Selectively Recognizes and Enters Tumor Cells

1) Preparation of diluted solution for B-phycoerythrin and R-phycoerythrin B-phycoerythrin and R-phycoerythrin (provided by Far East Bio-Tec Co., Ltd.) are to be passed through a sterile 0.22 um filter and the protein concentration are verified via respective absorption spectrum, and then serial dilution is carried out by using culture medium as diluent to obtain R-phycoerythrin diluted solutions of concentrations between 0.5 to 40 ug/ml and is stored under 4° C.

2) Selection of Normal and Tumor Cells

Human peripheral blood cells and fibroblast; rodent spleen cells, aster brain cells and fibroblast; plus various tumor cell lines (including cells from leukemia, hepatoma, lung cancer, thyroid cancer, gastric cancer, cervical cancer and brain cancer.)

3) Preparation of Cells

Most normal cells and tumor cells, which are anchorage dependent cell lines, are to be cultured in Dulbecco's Modified Eagle's Medium (DMEM) (pH7.4) supplemented with 10% Fetal Bovine Serum (FBS) under 37° C., 5% CO₂ and 95% saturated humidity. Medium is changed every other day. Upon subculturing the cells, old medium is discarded first and cells are washed once with phosphate buffered saline (PBS), then 0.25% trypsin-EDTA is added to detach cells from culture flask. Appropriate amount of above mentioned medium is then added to terminate the function of trypsin and to obtain cell suspension. Suspension cells (non-anchorage dependent cells) such as normal peripheral blood cells and leukemia cells are cultured in RPMI 1640 medium supplemented with 10% FBS (pH 7.4) under the same condition and the medium are to be replaced every other day. Upon subculturing the cells, cell suspension are only washed once with PBS.

4) Affinity Test

Take half million cells from above step (3) and incubate with various concentration of B-phycoerythrin or R-phycoerythrin for 0.5 hour or 24 hours, respectively. Use flow cytometry with the excitation wavelength at 488 nm so as to assess the amount of phycoerythrin entering cells.

5) Flow Cytometry Analysis:

Count cell numbers first, since 5-20*10⁵ cells are needed for each analysis. Wash cell suspension twice with PBS and then remove supernatant. Disperse cells and add 1 ml PBS buffer. Then, wrap the culture dish with aluminum foil to avoid light. Add 50-100 ng/ml PI to run on flow cytometry.

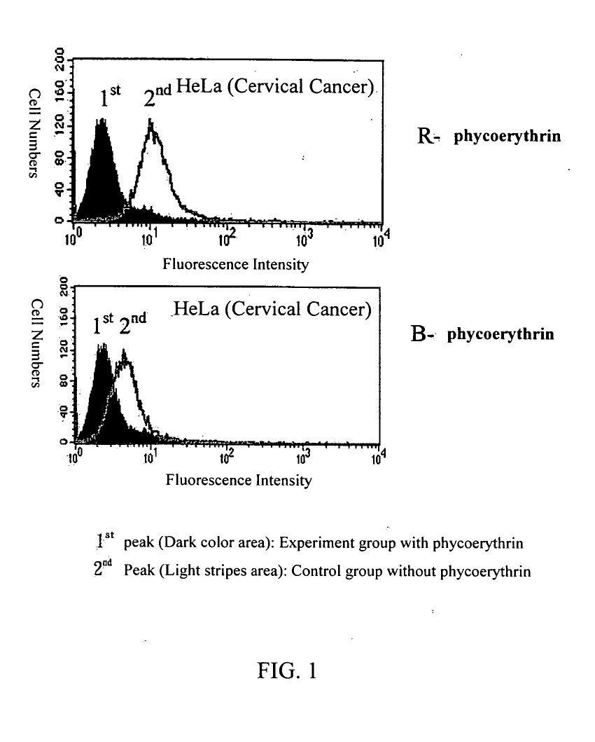

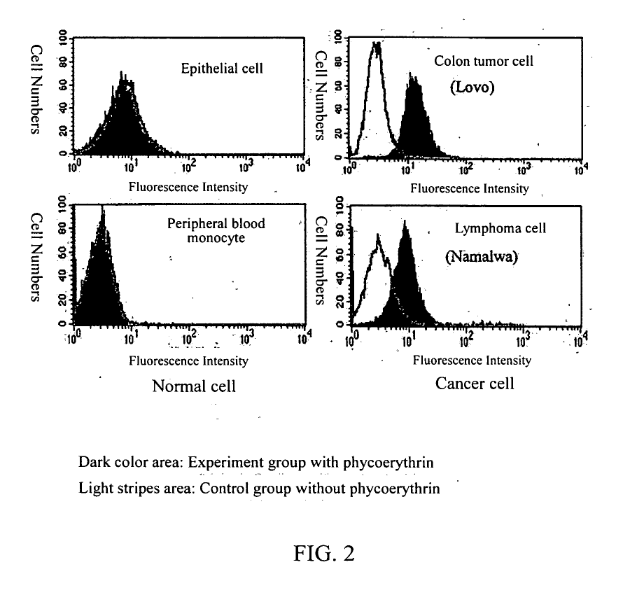

The result is shown in FIG. 1. Peak 1^{st} is cervical carcinoma cells without phycoerythrin, peak 2^{nd} is cervical carcinoma cells with phycoerythrin. Both cells were observed a significant fluorescent enhancement on experiment group, which indicates R-phycoerythrin and B-phycoerythrin can successfully enter cervical carcinoma cells and therefore enhances the fluorescent intensity. FIG. 2 demonstrates selective affinity of phycoerythrin to cells. When testing normal cells (epithelial cells and peripheral blood monocytes), overlapping signals were observed on group with phycoerythrin (dark color area) and group without phycoerythrin (light stripes area), indicating no change on fluorescent intensity, which represents no reaction between normal cells and phycoerythrin. However, when testing cancer cells (colon cancer cells and lymphoma), significant signal shift was observed between reaction group (dark color area) and control group (light stripes area), which indicates phycoerythrin can enter tumor cells but not normal cells, and demonstrates its high specificity to tumor cells. To further verify whether phycoerythrin has a extensive tumor-recognition ability, table 2 shows the test results of a series of tumor cells and normal cells incubated with R-phycoerythrin and B-phycoerythrin for 0.5 hour and 24 hours. The table demonstrates that both R-phycoerythrin and B-phycoerythrin can enter into most of tested human and other mammalian tumor cells in relatively short of time but not into normal cells.

TABLE 2
Selectively tumor-recognizing and entering by phycoerythrin (+ indicates
positive phycoerythrin signal; − indicates no phycoerythrin signal;
+/− indicates weak signal; X means not tested).

| | | B-phycoerythrin | | | | R-phycoerythrin | | | |
|---|---|---|---|---|---|---|---|---|---|
| | | Low conc. | | High conc. | | Low conc. | | High conc. | |
| Type of | | 0.5 μg/ml | | 10 μg/ml | | 0.5 μg/ml | | 10 μg/ml | |
| cells | Cell lines | 30 min | 24 hrs | 30 min | 24 hrs | 30 min | 24 hrs | 30 min | 24 hrs |
| Human | Fibroblast | − | − | − | − | − | − | − | − |
| normal | 01888 | | | | | | | | |
| cell lines | Fibroblast | − | − | − | − | − | − | − | − |
| | 01869 | | | | | | | | |
| | Fibroblast | − | − | − | − | − | − | − | − |
| | Peripheral blood | − | − | − | + | − | − | − | + |
| | cell #1 | | | | | | | | |
| | Peripheral blood | − | +/− | +/− | +/− | − | − | +/− | + |
| | cell #2 | | | | | | | | |
| | Peripheral blood | − | +/− | − | + | − | +/− | + | + |
| | cell #3 | | | | | | | | |
| | Peripheral blood | − | − | − | +/− | X | X | X | X |
| | cell #4 | | | | | | | | |
| | Peripheral blood | − | − | − | + | X | X | X | X |
| | cell #5 | | | | | | | | |
| Tumor | Lukemia K562 | + | + | + | + | + | + | + | + |
| cell lines | Lukemia | + | + | + | + | X | X | X | X |
| | NAMALWA | | | | | | | | |
| | Lukemia | + | + | + | + | + | + | + | + |
| | Ramos | | | | | | | | |
| | Lukemia HEL | + | + | + | + | + | + | + | + |
| | Lukemia Jurkat T | + | + | + | + | + | + | + | + |
| | Gastric cancer | + | +/− | + | + | − | +/− | + | + |
| | AGS | | | | | | | | |
| | Gastric cancer | +/− | +/− | +/− | + | +/− | +/− | +/− | + |
| | Scm-1 | | | | | | | | |
| | Hepatoma | − | +/− | +/− | + | − | +/− | +/− | + |
| | HepG2 | | | | | | | | |
| | Colon cancer | − | +/− | +/− | + | − | − | +/− | + |
| | lovo | | | | | | | | |
| | Colon cancer | +/− | +/− | +/− | + | +/− | +/− | +/− | + |
| | WiDr | | | | | | | | |
| | Human | +/− | +/− | + | + | − | +/− | + | + |
| | embryonic | | | | | | | | |
| | rhabdomyosarcoma | | | | | | | | |
| | (RD p53 mutant) | | | | | | | | |
| | lung fibroma | X | X | X | + | X | X | X | X |
| | WI-38 | | | | | | | | |
| | Thyroid cancer | − | − | +/− | +/− | − | − | +/− | + |
| | CG1 | | | | | | | | |
| | Thyroid cancer | − | +/− | +/− | + | − | + | +/− | + |
| | CG3 | | | | | | | | |
| | Cervical cancer | − | +/− | +/− | + | − | +/− | − | + |
| | HeLa | | | | | | | | |
| | Cervical cancer | − | + | +/− | + | − | +/− | + | + |
| | SKGIIIa | | | | | | | | |
| Other | Rat brain | − | − | +/− | + | − | − | − | − |
| normal | astrocyte (RBA) | | | | | | | | |
| cells | Mouse fibroblast | − | − | − | − | − | − | − | − |
| | (3T3) | | | | | | | | |
| | Mouse spleen | − | +/− | − | + | − | +/− | + | + |
| | Cell #1 | | | | | | | | |
| | Mouse spleen | − | − | +/− | + | − | +/− | +/− | + |
| | cell #2 | | | | | | | | |
| | Mouse spleen | − | − | +/− | +/− | | | | |
| | cell #3 | | | | | | | | |
| Other | Rat brain tumor | +/− | + | + | + | + | + | + | + |
| tumor | C6 | | | | | | | | |
| cells | Mouse hepatoma | +/− | + | + | + | X | X | X | X |
| | M7L-1#0 | | | | | | | | |
| | Mouse hepatoma | + | + | + | + | X | X | X | X |
| | ML-1#5 | | | | | | | | |

### EXAMPLE 2

### Phycoerythrin Selectively Recognizes and Enters Tumor Cells from Clinical Specimen

1) Preparation of diluted solution for B-phycoerythrin and R-phycoerythrin is the same as above-mentioned protocol.

2) Source of specimen: normal blood and blood from leukemia patients were from Chang Gung Memorial Hospital (Linko).

3) Affinity test

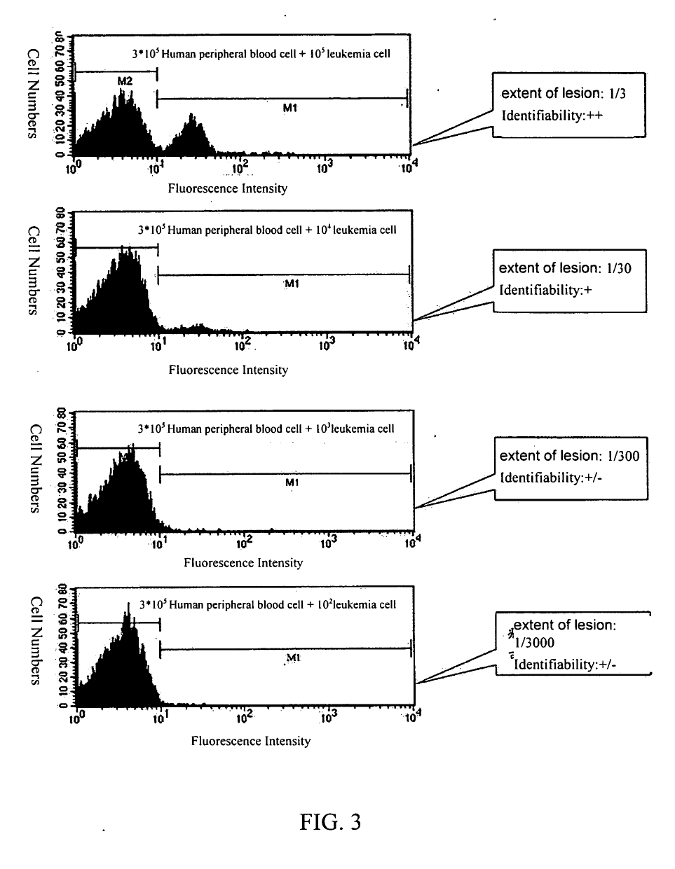

1 ml blood samples from various stages of leukemia were chosen, and the extent of lesion, namely the proportion of tumor cells to normal cells, is determined (from top to bottom): 1/3, 1/30, 1/300, 1/3000 via microscope. The blood samples are incubated with 2 μg/ml B-phycoerythrin or R-phycoerythrin (prepared from step 1) respectively for 30 minutes. Then the amount of phycoerythrin entering cells is monitored via flow cytometry with excitation wavelength at 488 nm to stimulate fluorescence of phycoerythrin. As shown in FIG. 3, the M1 area indicates tumor cells, and the M2 area indicates normal cells. The result indicates that the samples with larger extent of lesion (⅓ and 1/30) have larger M1 area, while the samples with smaller extent of lesion (1/300 and 1/3000) have insignificant M1 area. This data demonstrate that phycoerythrin has the capacity to label tumor cell and can be further utilized to verify various stages of tumor.

Taken together, the results of above examples show: 1) Phycoerythrin can selectively remain inside tumor cells; 2) Phycoerythrin can emit red fluorescence (the wavelength of fluorescence is at 575 nm) when illuminated with light at specific wavelength (from 488 nm to 633 nm, with the ideal wavelength being at 488 nm), which can be used to identify the location of tumor, its range and stage as well.

### EXAMPLE 3

### Observation of Phycoerythrin Entering Cells via Fluorescence Microscope.

1) Preparation of diluted solution for B-phycoerythrin is the same as above-mentioned protocol.

2) Observation of entering cells via fluorescence microscope

About half million hepatoma cells (Hep-G2) are seeded onto a appropriate carrier (i.e., cover slide), which is then placed in 24 well culture flask. After

16-hour incubation under above-mentioned condition, the medium is discarded and the cells are washed once with PBS, and then 1 ml R-phycoerythrin diluted solution prepared as above-mentioned is added. After 24 hour-incubation, the cells were washed three times with PBS, fixed with 4% Paraformaldehyde, and washed with PBS three times. The cells are harvested, embeded in 80% glycerol, and observed level of fluorescence staining under fluorescence microscope.

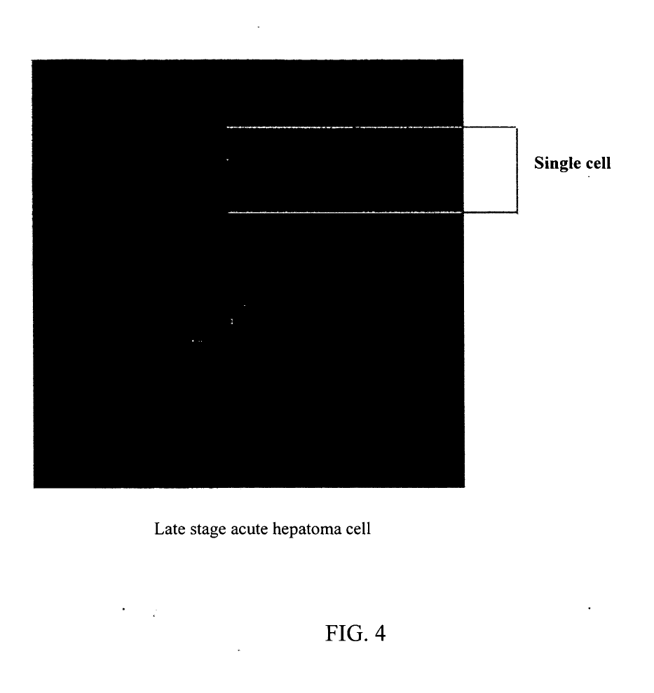

As shown in FIG. 4, one can observe significant fluorescent staining in tumor cells (white area), which demonstrates that B-phycoerythrin indeed can enter tumor cells.

The present invention, by utilizing phycoerythrin's high affinity to tumor cells that can be served to label tumor cells, provides a method for determining the location and range of tumor.

1. A method for labeling tumor tissues or cells by utilizing phycoerythrin comprises the steps of:
adding effective amount of phycoerythrin to tissues or cells and incubating for a certain period;
exposing said tissues or cells containing effective amount of phycoerythrin with appropriate wavelength of light to stimulate phycoerythrin to emit fluorescence; and
monitoring said fluorescence to identify the location and range of tumor tissues or cells.

2. The method as in claim 1, wherein said phycoerythrin comprises R-phycoerythrin or B-phycoerythrin.

3. The method as in claim 2, wherein said phycoerythrin is R-phycoerythrin.

4. The method as in claim 1, wherein the length of said certain period of incubation varies according to cell types, generally from 0.5 hour to 24 hours.

5. The method as in claim 1, wherein said wavelength of light is between 488 and 633 nm.

6. The method as in claim 2, wherein the wavelength for stimulating said R-phycoerythrin is at 488 nm.

7. The method as in claim 2, wherein the wavelength for stimulating said B-phycoerythrin is at 543 nm.

8. The method as in claim 1, wherein said effective amount of phycoerythrin is between 0.5 and 40 ug/ml.

9. The method as in claim 8, wherein said effective amount of phycoerythrin is between 0.5 and 10 ug/ml.

10. The method as in claim 1, wherein said fluorescence is monitored at wavelength above 565 nm.

11. The method as in claim 1, wherein said tumor can be solid or liquid tumor.

12. The method as in claim 11, wherein said tumor comprises: lymphoma, gastric cancer, hepatoma, colon cancer, lung fibroma, thyroid cancer, cervical carcinoma and brain tumor.

13. The method as in claim 1, wherein said labeling tumor tissues or cells means that the tumor-recognizing and tumor-entering ability of phycoerythrin is utilized in order to achieve the performance of labeling tumor tissues or cells.

14. A diagnostic kit for labeling tumor tissues or cells, comprising effective amount of phycoerythrin.

15. The diagnostic kit as in claimed 14, wherein said phycoerythrin comprises R-phycoerythrin or B-phycoerythrin.

16. The diagnostic kit as in claimed 14, wherein said phycoerythrin is R-phycoerythrin.

17. A method for labeling tumor tissues or cells by utilizing phycoerythrin comprises using the diagnostic kit according to claim 14.

18. The method as in claim 1, which can further comprises a step of analyzing the level of fluorescent staining of tumor so as to judge the stage of tumor.

19. The method as in claim 18, wherein said analyzing the level of fluorescent staining of tumor can be achieved through immunofluorescence staining or flow cytometry.
